(19) United States
(12) Patent Application Publication
(10) Pub. No.: US 2017/0136174 A1
(43) Pub. Date: May 18, 2017

(54) PORTABLE GAS DELIVERY SYSTEM

(71) Applicants: Frank Levy, Fort Myers, FL (US); Kimberley Levy, Fort Myers, FL (US)

(72) Inventors: Frank Levy, Fort Myers, FL (US); Kimberley Levy, Fort Myers, FL (US)

(21) Appl. No.: 14/944,574

(22) Filed: Nov. 18, 2015

(51) Int. Cl. A61M 5/00; A61M 39/22; A61M 5/19; A61M 13/00

(52) U.S. Cl. CPC A61M 5/007; A61M 13/003; A61M 39/223; A61M 5/19; A61M 2005/006

(57) ABSTRACT

A delivery system for the effective, reliable and foolproof delivery of controlled amounts of a medical grade gas to a patient includes a compressed gas unit composed of a vacuum sealed bag to which is secured at least one compressed gas cartridge and a multi-part valve delivery system.

### BACKGROUND OF THE INVENTION

1. Field of the Invention

This invention relates to a portable system for safely and efficiently producing and delivering CO₂ and other gases for use in medical applications.

2. Description of the Related Art

Conventional devices for delivering gas such as carbon dioxide (CO₂) for use in medical procedures typically utilize large storage tanks and regulators. Such devices are dangerous because of the risk of a seal, valve or part malfunction, which can produce a projectile in a medical setting. In addition, existing tank systems are quite expensive, extremely cumbersome and usually impractical to transport to off-site locations. These systems typically require a considerable amount of storage space. Current tanks also require filling at a filling station, which can involve the transport of a large quantity of gas such as CO₂. Pressurized gas tanks can explode in the event of a motor vehicle crash. Re-fellable tanks can also exhibit rust, bacteria and contamination, which are not acceptable in a medical environment.

Still further, various types of medical equipment have been utilized to deliver controlled volumes of liquid and gaseous substances to patients. One field that involves the administration of such fluids is radiology, wherein a small amount of CO₂ gas or an alternative contrast media may be delivered to the vascular system of the patient to displace the patient's blood and obtain improved images of the vascular system. Traditionally, this has required that the CO₂ or other media first be delivered from a pressurized cartridge to a syringe. The filled syringe is then disconnected from the cartridge and reconnected to a catheter attached to the patient. If additional CO₂ is needed, the syringe must be disconnected from the catheter and reattached to the cartridge for refilling. Not only is this procedure tedious and time consuming, it presents a serious risk of introducing air into the CO₂ or contrast fluid at each point of disconnection. Injecting such air into the patient's blood vessels can be extremely dangerous and even fatal.

Recinella et al., U.S. Pat. No. 6,315,762, discloses a closed delivery system wherein a bag containing up to 2,000 ml of CO₂ or other contrast media is selectively interconnected by a stopcock to either the chamber of a syringe or a catheter attached to the patient. Although this system does reduce the introduction of air into the administered fluid caused by disconnecting and reconnecting the individual components, it still exhibits a number of shortcomings. For one thing, potentially dangerous volumes of air are apt to be trapped within the bag. This usually requires the bag to be manipulated and flushed multiple times before it is attached to the stopcock and ultimately to the catheter. Moreover, this delivery system does not feature an optimally safe and reliable, foolproof operation. If the stopcock valve is incorrectly operated to inadvertently connect the CO₂ filled bag or other source of CO₂ directly to the patient catheter, a dangerous and potentially lethal volume of CO₂ may be delivered suddenly to the patient's vascular system. It is medically critical to avoid such CO₂ flooding of the blood vessels.

### SUMMARY OF THE INVENTION

It is therefore an object of the present invention to provide a delivery system for the effective, reliable and foolproof delivery of controlled amounts of a medical grade gas to a patient. The delivery system includes a compressed gas unit composed of a vacuum sealed bag to which is secured at least one compressed gas cartridge and a multi-part valve delivery system.

It is also an object of the present invention to provide a delivery system wherein the compressed gas cartridges are approximately 16 g to 45 g compressed gas cartridges.

It is another object of the present invention to provide a delivery system wherein the compressed gas is medical grade CO₂ gas.

It is a further object of the present invention to provide a delivery system wherein the multi-part valve delivery system includes a flow control system.

It is also an object of the present invention to provide a delivery system wherein the flow control system includes an inlet conduit for being communicably joined to the integrated compressed gas unit, an outlet conduit for being communicably joined to the patient, first and second syringes intermediate the inlet and outlet conduits, and a control valve assembly interconnecting the inlet conduit, the outlet conduit, the first syringe and the second syringe.

It is another object of the present invention to provide a delivery system wherein the control valve assembly is alternatable between a first state wherein the inlet conduit communicates with the first syringe for transmitting fluid from the source to only the first syringe, a second state wherein the first syringe communicates only with the second syringe and is isolated from the inlet and outlet conduits for transmitting fluid from the first syringe to only the second syringe, and a third state wherein the second syringe communicates only with the outlet conduit and is isolated from the inlet conduit and the first syringe for transmitting fluid from the second syringe to only the outlet conduit.

It is a further object of the present invention to provide a delivery system wherein the control valve assembly includes a valve body having aligned inlet and outlet ports. The inlet port is communicably connectable to the inlet conduit and the outlet port being communicably connectable to the outlet conduit. The valve body further includes a first intermediate port to which the first syringe is selectively connected and a second intermediate port to which the second syringe is selectively connected. The control valve assembly further includes a stopcock element mounted rotatably within the body and including a channel consisting essentially of a first channel segment and a second channel segment. The first and second channel segments are selectively alignable with the inlet port and the first intermediate port to allow for communication between the inlet conduit and the first syringe, the first intermediate port and the second intermediate port to allow for communication between the first syringe and the second syringe. The second intermediate port and the outlet port allow for communication between the second syringe and the outlet conduit.

It is also an object of the present invention to provide a delivery system wherein the sealed bag includes a central cavity, an inlet port and an outlet port.

It is another object of the present invention to provide a delivery system wherein the sealed bag includes a loop allowing the sealed bag to be conveniently supported by a conventional medical stand.

It is a further object of the present invention to provide a delivery system wherein the inlet port includes a cylinder cartridge puncture valve.

It is also an object of the present invention to provide a delivery system wherein the outlet part is connected to the multi-part valve delivery system.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 is a schematic of the present portable gas delivery system.

FIG. 2 is a detailed top view of the control valve assembly shown in FIG. 1.

FIGS. 2A, 2B and 2C show the various positions in which the stopcock may be placed in accordance with the present invention.

FIG. 3 is a schematic view of the outlet conduit and alternative downstream fittings that may be used to interconnect the outlet conduit to the patient.

FIG. 4 is a schematic depicting a medication administering syringe being attached to the downstream fitting via a connecting tube.

FIG. 5 is a perspective view of an alternate control valve assembly in accordance with the present invention.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as a basis for teaching one skilled in the art how to make and/or use the invention.

The present invention provides a delivery system 10 for the effective, reliable and foolproof delivery of controlled amounts of a medical grade gas, in particular, CO₂ or other contrast media, to a patient. In accordance with the present invention, delivery is achieved through the utilization of a compressed gas unit 12 and a multi-part valve delivery system 14. The multi-part valve delivery system 14 delivers the medical grade gas in precisely controlled amounts sequentially through a series of syringes such that it is impossible to directly connect the source to the patient. At the same time, the delivery system 10 does not have to be disconnected and reconnected during the administration of medical grade gas. This greatly reduces the intrusion of air into the system and the fluid being administered.

The present invention is intended to provide a portable, safe, reliable, and convenient source of medical gas, such as medical grade CO₂ to health care professionals in hospital or medical office settings where a small volume of CO₂ or other gases is needed. The present invention is simple to manufacture and use because it does not require large regulators, an external power source, cumbersome large tanks or impellers for dispensing medical grade CO₂. The present invention utilizes a fluid source in the form of small size compressed gas cartridges 18 of approximately 16 g to 45 g to produce the desired pressure and airflow for the effective transformation of medical grade CO₂ liquid to medical grade CO₂ gas. The compressed gas is contained in one or more compressed gas containers such as cylindrical cartridges. As those skilled in the art will appreciated given the transformation of from liquid to gas as achieved when employing compressed gas cartridges, the term “fluid” should be understood to include various types of medical liquids and gases. By the same token, when “gas” is used herein, it should be understood that such description is likewise applicable to various types of medical liquids.

The delivery system 10 of this invention is particularly beneficial for delivering CO₂ for medical use. In medical uses, CO₂ is used because it is safer and has fewer complications than air or oxygen in the same uses. CO₂ diffuses more naturally in body tissue and is absorbed in the body more rapidly with fewer side effects. CO₂ used in decompartmentalization of tissues, arteries, veins and nerves and for radiological imaging, cardiac imaging, evaluation of vascularity of the heart and surrounding tissues, oncology and urology diagnostics. It is specifically used for imaging by infiltrating the tissues, body cavities and abdomen for better visualization. The CO₂ can also expand internal body cavities and tissues thereby enabling better diagnostic techniques.

The CO₂ gas provided by the compressed gas unit 12 of this invention is ultimately delivered to a flow control system 60 of the multi-part valve delivery system 14. As will be explained below in greater detail, the flow control system 60 provides a foolproof mechanism for the delivery of CO₂ to be used as needed by various medical devices for applications such as imaging, differentiation of tissues, arterial/venus/neurological separation, and treatment of stretch marks, facial wrinkles and dark circles.

The delivery system 10 is portable and compact so that it is convenient to utilize in the field for portable medical uses, military field uses and any other use requiring CO₂ or other medical gas for its performance. The delivery system 10 is safer than existing tank systems because it eliminates the risk of seal, valve or part malfunction, the potential for a disastrous explosion and the unwanted production of projectiles in a medical setting. It also eliminates the rust, dirt, bacteria and contaminants that can be present in refillable tanks. The delivery system 10 requires very little space to store and is much easier to use. The delivery system 10 of the invention is much less expensive than current tank systems. In addition, the delivery system 10 utilizes compact compressed gas cartridges 18 which can be delivered and transported in a small box. The compressed gas cartridges 18 do not have to be transported to a filling station and do not present a risk of explosion in the event of a motor vehicle accident.

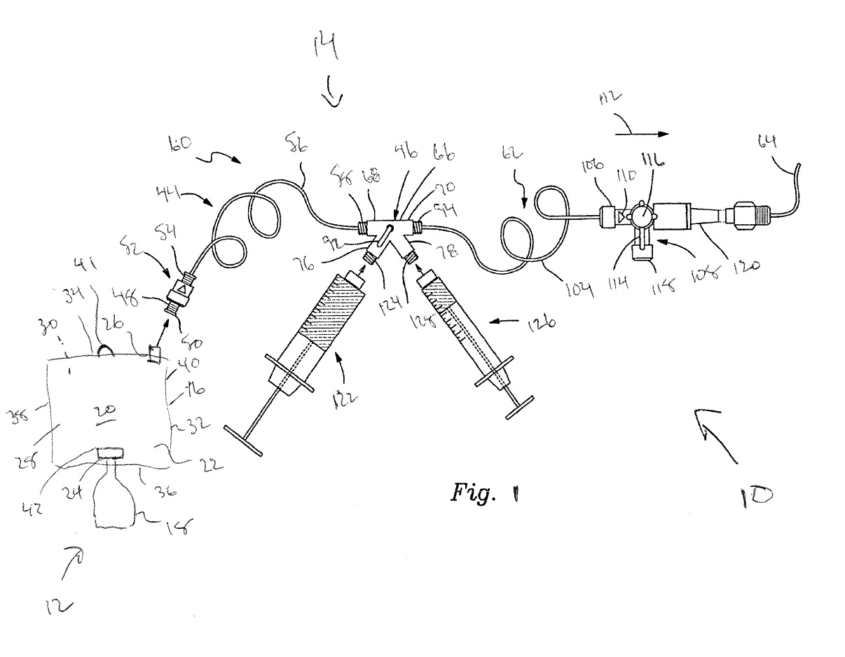

Referring now to FIG. 1, the compressed gas unit 12 of the present invention is disclosed. The compressed gas unit 12 includes a vacuum sealed bag 16 to which is secured at least one compressed gas (CO₂) cartridge 18. As will be explained below in greater detail, the sealed bag 16 includes an outlet that is in fluid communication with the multi-part valve delivery system 14.

In accordance with a preferred embodiment, the sealed bag 16 is vacuum sealed and is composed of a polyurethane film. The sealed bag 16 includes a central cavity 20 defined by a bag wall 22, which is fully sealed with the exception of an inlet port 24 and an outlet port 26. Although it is appreciated various bag construction are possible in accordance with the present invention, the present sealed bag 16 includes a front first wall 28 and a rear second wall 30. The first and second walls 28, 30 are coupled along their respective edges to define a sealed edge 32 of the sealed bag 16 and to create the central cavity 20 of the sealed bag 32. The sealed edge 32 includes a top edge 34, a bottom edge 36 and first and second side edges 38, 40 extending between the top edge 34 and the bottom edge 36. The sealed bag 16 is also provided with a loop 41 along the top edge 34 allowing the sealed bag 16 to be conveniently supported by a conventional medical stand.

As briefly discussed above, the sealed bag 16 is provided with an inlet port 24 and an outlet port 26, and these ports are respectively integrated into the sealed edge 32. In particular, and in accordance with a preferred embodiment, the inlet port 24 is integrated into the bottom edge 36 of the sealed bag 16 and the outlet port 26 is integrated into the top edge 34 of the sealed bag 16.

The inlet port 24 is shaped and dimensioned for selective attachment of the compressed gas cartridge 18 for the passage of gas from the compressed gas cartridge 18 into the central cavity 20 of the sealed bag 16. The inlet port 24 includes a cylinder cartridge puncture valve 42. The cartridge puncture valve 42 is constructed with a one-way valve permitting the flow of compressed gas into the sealed bag 16 but preventing the gas from flowing out of the sealed bag 16 through the cartridge puncture valve 42. The cartridge puncture valve 42 has a mechanism for piercing the compressed gas cartridge 18, as is known in the art, and for holding or securing the compressed gas cartridge 18 in place (for exampling, via mating threading formed along the neck of the compressed gas cartridge 18 and the cartridge puncture valve 42). As such, and once the compressed gas cartridge 18 is puncture, the complete contents of the compressed gas cartridge will flow into the sealed bag 16. The sealed bag 16 is, therefore, sized to receive the entire content of the compressed gas cartridge 18.

The outlet port 26 is shaped and dimensioned for attachment to the multi-part valve delivery system 14. In accordance with a preferred embodiment attachment is achieved through the provision of an inlet conduit 44 communicably interconnecting the central cavity 20 of the sealed bag 16 to the control valve assembly 46 of the multi-part valve delivery system 14. The inlet conduit 44 includes a first Luer fitting 48 having a G-tube seal 50, which is selectively attached to the outlet port 26 of the sealed bag 16. A one-way directional valve 52 with a second Luer fitting 54 is communicably joined to the first Luer fitting 48. The second Luer fitting 54 is, in turn, communicably joined to a coiled medical tube 56 having a length of approximately 18″. Various alternative lengths may be employed within the scope of this invention. The distal end of the tube 56 carries a third Luer fitting 58 that is selectively secured to the control valve assembly 46.

As briefly explained, the delivery of CO₂ as described above is facilitated by the provision of a multi-part valve delivery system 14. As briefly mentioned above, the multi-part valve delivery system 14 includes a flow control system 60 as disclosed with reference to FIGS. 1 to 5 and as described in U.S. Patent Application Publication No. 2012/0065502, which is incorporated herein by reference.

The flow control system 60 provides an improved, foolproof mechanism for safely delivering controlled amounts of a medical fluid such as CO₂ or other contrast media to a patient by utilizing a multi-part valve that delivers the fluid in precisely controlled amounts sequentially through a series of syringes such that it is impossible to directly connect the gas source to the patient. At the same time, the delivery system does not have to be disconnected and reconnected during administration of the medical grade gas. This greatly reduces the intrusion of air into the system and the fluid being administered.

The flow control system 60 provides for controlled delivery of a medical grade gas from a compressed gas cartridge to a patient. As will be explained below in greater detail, the flow control system 60 includes the previously discussed inlet conduit 44 that is communicably joined to the sealed bag 16 and an outlet conduit 62 that is communicably joined to the patient. First and second syringes 122, 126 are intermediate the inlet and outlet conduits 44, 62. A control valve assembly 46 of the flow control system 60 interconnects the inlet and outlet conduits 44, 62 as well as the intermediate first and second syringes 122, 126. The control valve assembly 46 is alternatable between first, second, and third states. In the first state, the inlet communicates with the first syringe 122 for transmitting gas from the compressed gas cartridge 18 to the first syringe 122. In the second state, the first syringe 122 communicates with the second syringe 126 and is isolated from the inlet and the outlet conduits 44, 62 for transmitting fluid from the first syringe 122 to the second syringe 126. In the third state, the second syringe 126 communicates with the outlet conduit 62 and is isolated from the inlet conduit 44 and the first syringe 122. This allows fluid to be transmitted from the second syringe 126 to the patient through the outlet conduit 62.

In one embodiment, the control valve assembly 46 includes a valve body 66 having aligned inlet and outlet passageways that are communicably connectable to the inlet and outlet conduits 44, 62 respectively. The valve body 66 further includes a pair of first and second transverse passageways that extend axially transversely to the inlet and outlet passageways and transversely to each other. A stopcock 84 is mounted rotatably within the valve body 66 and includes an angled stopcock channel 86 having a pair of communicably interconnected channel segments 88, 90 that extend axially at an acute angle to one another. The channel segments 88, 90 of the stopcock 84 are interconnected at an angle that is generally equivalent to the angle formed between each adjacent pair of non-aligned passageways in the valve body 66 such that the stopcock 84 is rotatable to align the channel segments 88, 90 with a selected adjacent pair of the non-aligned passageways to permit fluid communication between those passageways. Each of the transverse passageways is connectable to a respective syringe 122, 126. The stopcock 84 is selectively adjusted between first, second and third positions. In the first position, the channel segments 88, 90 communicably interconnect the inlet passageway 72 and the first transverse passageway 80. Fluid introduced through the inlet conduit 44 is thereby transmitted through the inlet passageway 72 and the angle stopcock channel 86 to the first transverse passageway 80. The first transverse passageway 80 directs the fluid to the first syringe 122 attached thereto. In the second valve position, the stopcock 84 aligns the channel segments 88, 90 with the first and second transverse passageways 80, 82 respectively. This isolates the fluid in the first syringe 122 from both the inlet and outlet conduits 44, 62. The first syringe 122 is operated to direct the fluid through the first transverse passageway 80, the angled stopcock channel 86 and the second transverse passageway 82 into a second syringe 126 joined to the second transverse passageway 82. In the third valve position, the stopcock 84 is rotated to align the channel segments 88, 90 with the second transverse passageway 82 and the outlet passageway 74 respectively. This isolates the gas in the second syringe 126 from the compressed gas cartridge 18, the inlet passageway 72 and the first transverse passageway 80. The second syringe 126 is then operated to drive the gas through the second transverse passageway 82, the angled stopcock channel 86 of the stopcock 84 and the outlet passageway 74 to the outlet conduit 62. The outlet conduit 62 directs this fluid to the patient.

The respective longitudinal axes of the inlet and outlet passageways 72, 74 are aligned. The first and second transverse passageways 80, 82 include respective longitudinal axes that form an angle of substantially 60 degrees with one another. The first transverse passageway 80 forms an axial angle of substantially 60 degrees with the longitudinal axis of the inlet passageway 72 and, similarly, the axis of the second transverse passageway 82 forms an angle of substantially 60 degrees with the longitudinal axis of the outlet passageway 74.

The angled stopcock channel 86 formed in the stopcock 84 preferably features channel segments 88, 90 with respective longitudinal axes that form an angle of substantially 60 degrees. Alternative angles may be featured when the inlet and outlet conduits 44, 62 are not aligned.

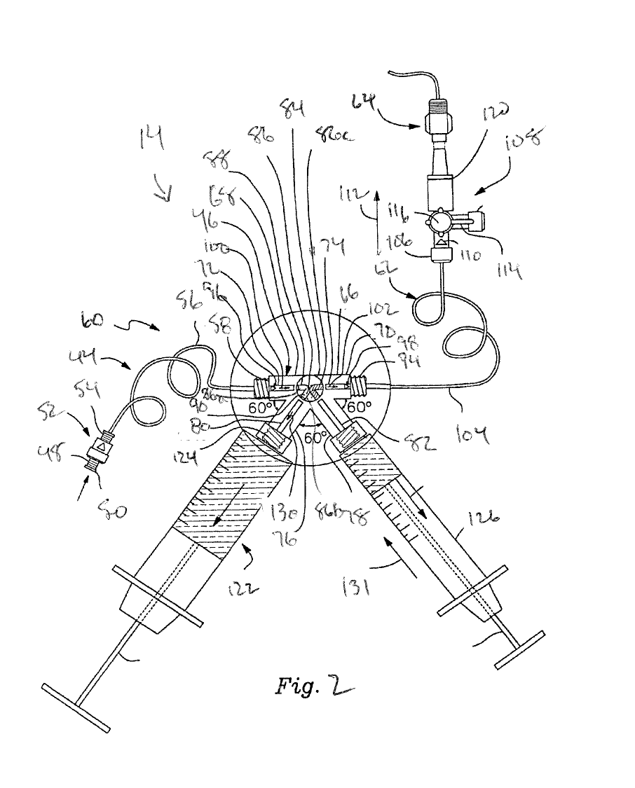

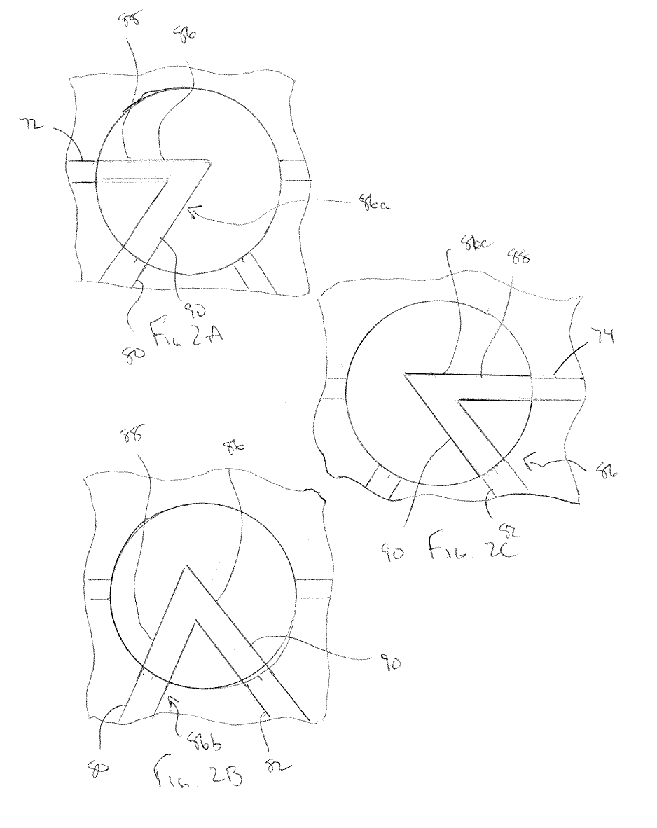

Referring to FIGS. 1 and 2 the flow control system 60 for delivering controlled dosages of a medical contrast fluid such as CO₂ for use in the radiological imaging of arteries and veins of a patient's vascular system is shown in detail. Although this is a preferred application for the flow control system 60, it should be understood that the flow control system 60 may be used for the controlled delivery of various other types of liquids and gases administered as part of assorted surgical and medical procedures.

The flow control system 60 includes an inlet conduit 44 and an outlet conduit 62 interconnected by the three-stage K-valve shaped control valve assembly 46. The inlet conduit 44 communicably interconnects a fluid source, for example, pressurized CO₂, from the compressed gas unit 12 with the control valve assembly 46. The outlet conduit 62 likewise communicably interconnects a discharge end of the control valve assembly 46 with a catheter 64 that is, in turn, operably connected to a patient, not shown.

As explained above, the inlet conduit 44 includes the first Luer fitting 48 having the G-tube seal 50, which is selectively attached to the compressed gas cartridge 18. A one-way directional valve 52 with the second Luer fitting 54 is communicably joined to the first Luer fitting 48. The second Luer fitting 54 is, in turn, communicably joined to the coiled medical tube 56 having a length of approximately 18″. Various alternative lengths may be employed within the scope of this invention. The distal end of the tube 56 carries a Luer fitting 58.

The three-stage control valve assembly 46 includes a generally K-shaped valve body 66, which is preferably composed of various medical grade plastics, metals and/or metal alloys. Typically, the valve body 66 includes a molded or otherwise unitary construction. More particularly, the valve body 66 includes aligned intake and discharge branches 68 and 70, respectively, which, as best shown in FIG. 2, include respective aligned internal inlet and outlet passageways 72, 74. The valve body 66 also includes first and second transverse valve legs 76, 78. Each valve leg 76, 78 extends at an angle of substantially 60 degrees from aligned intake and discharge branches 68, 70 of the valve body 66. The first valve leg 76 includes an interior first transverse passageway 80 and the second valve leg 78 includes an interior second transverse passageway 82, which extend axially longitudinally through the respective first and second valve legs 76, 78. The first and second passageways 80, 82 form angles of substantially 60 degrees apiece with the respective axial inlet and outlet passageways 72, 74 of the aligned intake and discharge branches 68, 70. The transverse first and second valve legs 76, 78 also extend at an angle of substantially 60 degrees to one another. By the same token, the longitudinal axes of the first and second transverse passageways 80, 82 form an angle of substantially 60 degrees.

The control valve assembly 46 further includes a stopcock 84 that, as best shown in FIG. 2, is rotatably mounted within valve body 66. The stopcock 84 includes the angled stopcock channel 86 comprising communicably interconnected channel segments 88 and 90 having respective longitudinal axes that extend at an angle of approximately 60 degrees to one another. As used herein, “approximately 60 degrees” should be understood to mean that the angle formed between the respective longitudinal axes of the channel segments 88, 90 is substantially equivalent to the angle formed between the longitudinal axes of respective pairs of the non-aligned adjacent passageways of valve body 66 (e.g. respective pairs of passageways 72, 80; 80, 82; and 82, 74). This enables the channel segments 88, 90 to be communicably aligned with a selected pair of the passageways 72, 74, 80, 82 in the manner described more fully below. It should be understood that in alternative embodiments the passageways and channel segments may have other corresponding angles.

As shown in FIG. 1, a valve lever 92 is mounted to the stopcock 84 within valve body 66 for selectively rotating the stopcock 84 into a selected one of three positions. For example, in FIG. 2, the stopcock 84 is positioned with the channel segments 88, 90 of the angled stopcock channel 86 communicably aligned with adjacent inlet and first transverse passageways 72, 80, respectively (see FIG. 2A). Alternately, and as described more fully below, the lever 92 may be manipulated to align the channel segments 88, 90 with respective first and second transverse passageways 80 and 82 as indicated by the channel shown in phantom in position 86b (see FIG. 2B). The lever 92 may be likewise operated to align the respective channel segments 88, 90 with the second transverse and outlet passageways 82, 74 as indicated by the angled channel in position 86c (see FIG. 2C). Such selective positioning of the stopcock 84 provides for controlled multiple stage delivery of gas through the control valve assembly 46 from the inlet conduit 44 to the outlet conduit 62. This operation is described more fully below.

The intake branch 68 of the valve body 66 carries a complementary fitting for communicably interconnecting to the third Luer fitting 58 carried at the distal end of the tubing 56. By the same token, the discharge branch 70 of the valve body 66 carries a complementary fitting for operably and communicably interconnecting with a fourth Luer fitting 94 carried at the proximal end of the outlet conduit 62. The remaining elements of the discharge conduit are described more fully below. The aligned inlet and outlet passageways 72, 74 of the valve body 66 include respective one-way valves 96, 98, FIG. 2, which restrict or limit the flow of fluid within the respective inlet and outlet passageways 72, 74 to the direction indicated by arrows 100 and 102.

As further illustrated in FIGS. 1 and 2, the outlet conduit 62 features a coiled medical tube 104 that is communicably interconnected between the fourth Luer fitting 94 attached to the discharge branch 70 of the valve body 66 and a fifth Luer fitting 106, which is communicably joined to a downstream valve 108. The downstream valve 108 includes a one-way valve 110 that restricts fluid flow from the outlet conduit 62 through the downstream valve 108 to the direction indicated by arrow 112. The downstream valve 108 features a G-tube seal 114 that prevents air from intruding into the system prior to connection of the downstream valve 108. The downstream valve 108 also includes a stopcock 116 that is rotatably operated within the downstream valve 108 to selectively bleed or purge fluid from the flow control system 60 through a port 118. Exit port 120 is selectively joined to the patient catheter 64. Various alternative two and three port stopcocks may be used in the downstream valve.

A reservoir syringe 122 is communicably connected to the axial passageway 80 of the first valve leg 76. Such interconnection is accomplished by a conventional sixth Luer fitting 124, the details of which will be known to persons skilled in the art. Similarly, a second, draw-push syringe 126 is releasably attached by seventh Luer fitting 128 to the distal end of the second valve leg 78. This allows the second syringe 126 to be communicably interconnected with the second transverse passageway 82 through the second transverse valve leg 78. The first and second syringes 122 and 126 are constructed and operated in a manner that will be known to persons skilled in the art.

The flow control system 60 is operated to deliver CO₂ or other medical fluid to a patient in a controlled and extremely safe and reliable manner. This operation is performed as follows.

The inlet conduit 44 is first interconnected between a source of CO₂ via the compressed gas unit 12 in the form of the sealed bag 16 and the intake branch 68 of the valve body 66. The outlet conduit 62 likewise is communicably interconnected between the discharge branch 70 of the valve body 66 and the downstream valve 108, which is itself attached to the patient catheter 64. The first and second syringes 122, 126 are joined to the first and second transverse valve legs 76, 78 such that the first and second syringes 122, 126 communicate with the respective first and second transverse passageways 80, 82. The syringes should be selected such that they have a size that accommodates a desired volume of gas to be administered to the patient during the radiological imaging or other medical/surgical procedure.

After multistage K-valve control assembly 46 has been interconnected between the inlet conduit 44 and the outlet conduit 62, and following attachment of the first and second syringes 122, 126 to the respective first and second transverse valve legs 76, 78, the stopcock 84 is operated by the valve lever 92 to align the channel segments 88, 90 of the stopcock channel 86 with the inlet and first transverse passageways 72, 80 respectively. See FIG. 2. The compressed gas cartridge 18 is then connected to the input port 24 of the sealed bag 16 releasing the gas from the compressed gas cartridge 18 and into the sealed bag 16, which will then inflate with medical grade gas. The gas may then be delivered from the sealed bag 16, through the inlet conduit 44 to the control valve assembly 46. More particularly, and with reference to FIGS. 2 and 2A, the gas is delivered through the one-way valve 52 and the tubing 56 to the inlet passageway 72. The one-way valve 96 prevents backflow of gas into the coil tubing 56. The CO₂ proceeds in the direction indicated by arrow 100 and is transmitted through the angled stopcock channel 86 into the passageway 80 of the first valve leg 76. From there, the gas proceeds as indicated by arrow 130 through the fitting 124 and into the reservoir first syringe 122. The CO₂ is introduced into the reservoir first syringe 122 in this manner until it fills the syringe.

When the reservoir first syringe 122 is filled, the operator manipulates lever 92, FIG. 1, and rotates the control valve into the second stopcock channel position represented in phantom by 86b in FIG. 2 (and as shown in FIG. 2B). In that position, the channel segment 88 is communicably aligned with the passageway 80 and the channel segment 90 is communicably aligned with the passageway 82. The plunger 132 of the reservoir first syringe 122 is pushed and the gas previously deposited into the reservoir first syringe 122 is transmitted through the first transverse passageway 80 and the angled stopcock channel 86 into the second transverse passageway 82. From there, the gas is introduced into draw-push syringe 126 as indicated by arrow 134. As this operation occurs, only the first and second transverse passageways 80, 82 and their attached syringes 122, 126 are communicably connected. Both syringes 122, 126 remain completely isolated from both the inlet passageway 72 and the outlet passageway 74. By the same token, the source of CO₂ and communicably joined inlet passageway 72 are isolated from the outlet passageway 74 and the outlet conduit 62 connected to the catheter 64. The patient is thereby safely protected against being inadvertently administered a dangerous dosage of CO₂ directly from the source.

After the gas is transferred from the reservoir first syringe 122 to the push-draw second syringe 126, the operator manipulates the valve lever 92 to rotate the stopcock 84 to the third position, which is represented by the stopcock channel in position 86c (and as shown in FIGS. 2 and 2C). Therein, the channel segment 88 is communicably aligned with the second transverse passageway 82 and the channel segment 90 is similarly aligned with the outlet passageway 74. To administer the CO₂ in the second syringe 126 to the patient, the plunger 108 of the second syringe 126 is depressed in the direction of arrow 131. Gas is thereby delivered through the second transverse passageway 82 and the angled stopcock channel 86 into the outlet passageway 74. From there, the gas passes in the direction indicated by arrow 102 through one-way valve 98 and into tubing 104. CO₂ is thereby transmitted in the direction indicated by arrow 102 through the one-way valve 98 and into the tubing 104 of the outlet conduit 62. The one-way valve 98 prevents backflow of gas into the K-control valve assembly 46.

The lever 92 may be configured as an arrow or otherwise marked to include an arrow that points in the direction of the intended fluid flow. With the lever pointing toward the reservoir first syringe 122, as shown in FIG. 1, the angled stopcock channel 86 is in the position 86a shown in FIGS. 2 and 2A and fluid flow is directed toward the reservoir first syringe 122. Alternatively, the lever 92 may be rotated to point toward the second syringe 126. In this position, the angled stopcock channel 86 is in the position 86b shown in FIGS. 2 and 2B and CO₂ is directed from the first syringe 122 to the second syringe 126. Finally, in the third stage of the process, the lever 92 may be directed to point toward the discharge end of the outlet passageway 74 and the attached outlet conduit 62. In this stage, angled stopcock channel 86 is directed to the position 86c, shown in FIGS. 2 and 2C, such that fluid flow is directed from second syringe 126 to the outlet conduit 62.

CO₂ is delivered through the tube 104 and into the downstream valve 108. Once again, a one-way valve 110 prevents the backflow of gas into the tubing. The stopcock 116 is operated, as required, to either direct the CO₂ to the catheter 64 and thereby to the patient, or to purge the gas through port 118. The G-tube seal 114 prevents air from entering the line.

Accordingly, the flow control system 60 enables controlled amounts of CO₂ to be delivered to the patient in a safe and reliable manner. After the components are connected, they may remain connected during the entire medical procedure and do not then have to be disconnected and reconnected. This minimizes the possibility that air will intrude into the system and endanger the patient. Controlled and precise dosages of CO₂ are delivered, by the simple and foolproof operation of the control valve assembly 46, from the reservoir first syringe 122 to the push-draw second syringe 126 and then to the patient. At each stage of the process, the inlet and outlet ends of the valve remain totally isolated from one another so that the risk of administering an explosive and potential deadly dose of CO₂ is eliminated.

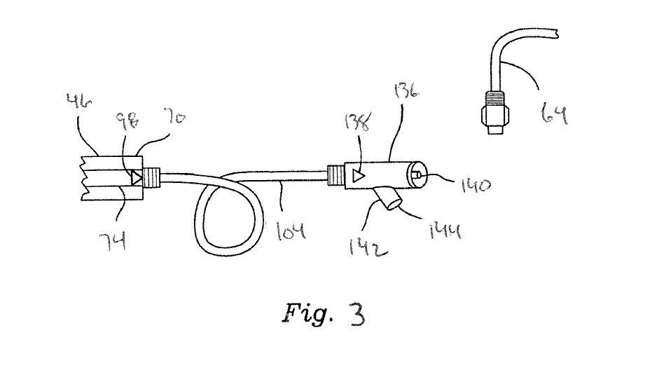

FIG. 3 illustrates the discharge branch 70 of the control valve assembly 46. A one-way valve 98 is again installed in the outlet passageway 74 to prevent backflow of gas into the control valve assembly 46. In this version, the tube 104 is communicably connected between the discharge branch 70 and a fitting 136 that may be used selectively to perform various functions. In particular, the fitting 136 includes a one-way valve 138 that prevents backflow of gas into the tube 104. The fitting 136 includes a Luer fitting 140 that allows the fitting 136 to be releasably attached to the catheter 64. A flush port 142 is communicably joined with the fitting 136 and features a G-valve seal 144 that permits a syringe (not shown) to be interconnected to the port 142. This syringe may be used to administer medications through the fitting 136 to the attached catheter 64. As a result, such medications may be administered to the patient without having to disconnect the individual components of the fluid delivery system. This saves valuable time in a surgical or medical environment and reduces the risk that air will be introduced into the system. A syringe may also be attached to port 142 to purge or flush the catheter as needed or desired.

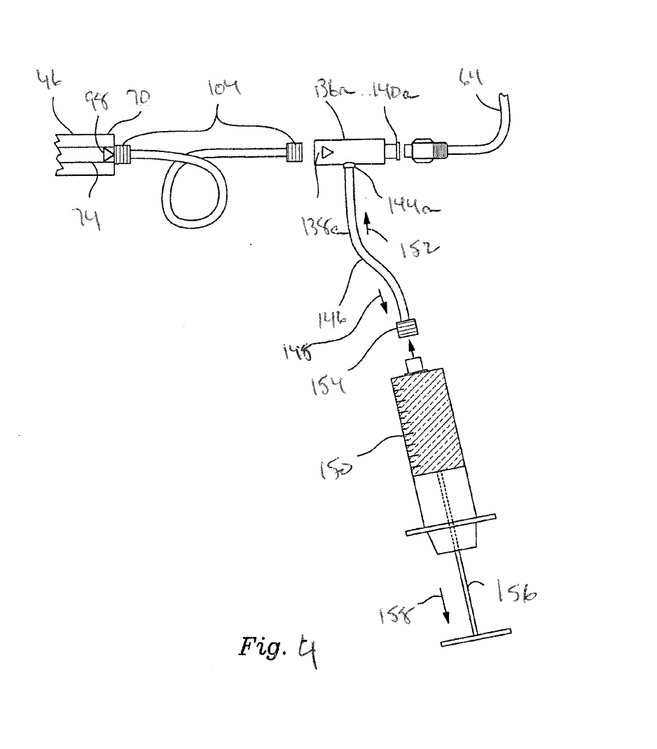

FIG. 4 depicts still another embodiment of this invention wherein the medical tube 104 is communicably interconnected between the discharge branch 70 of the control valve assembly 46 and a fitting 136a. The downstream fitting again includes a one-way valve 138a for preventing the backflow of gas or medication into the tube 104. A Luer fitting 140a releasably interconnects the fitting 136a to the catheter 64. An inlet/discharge port 144a is formed in the fitting 136a for selectively introducing medication into the patient catheter through the fitting 136a or alternatively purging or flushing the catheter as required. A line 146 is communicably connected to port 144a and carries at its opposite end a Luer fitting 148 for releasably attaching the line to a syringe 150. The syringe 150 is attached to the line 136 through the fitting 148 in order to optionally deliver medication to the catheter 64 through the fitting 136a in the direction indicated by arrow 152. Alternatively, fluid may be purged or flushed in the direction of arrow 154 from the catheter and/or from the system through the line 146 by drawing the plunger 156 of the syringe 150 rearwardly in the directions indicated by arrow 158.

In alternative versions of this invention, medical fluid may be transmitted from a source to a patient in multiple stages, as described above, but utilizing multiple valves joined to respective syringes. In particular, in a first stage operation, gas or other fluid under pressure is delivered from the source through a first directional valve to a reservoir syringe communicably connected to the first valve. The reservoir syringe is also connected through the first valve to a second valve which is, in turn, communicably joined to a second syringe. The first valve is operated so that the reservoir syringe remains isolated from the second valve as fluid is delivered from the source to the first syringe through the first valve. When a selected volume of fluid is accommodated by the first syringe, the first valve is operated to connect the first syringe with the second valve. The second valve itself is operated to communicably connect the first syringe to the second syringe while, at the same time, isolating the second syringe from the patient. The second syringe is a push-draw syringe. The first syringe is operated with the second valve in the foregoing position to transmit the fluid from the first syringe to the second syringe. During this stage of the operation, both syringes remain isolated from the source and the patient. As a result, even if fluid under pressure is “stacked” in the reservoir syringe, this pressure is not delivered to the patient. Rather, the desired volume of the fluid is delivered instead to the push-draw syringe. The second valve is then operated to communicably join the push-draw syringe to the patient and/or patient catheter. Once again, the patient and/or patient are totally isolated from the source of fluid under pressure. As a result, a safe and selected volume of fluid is delivered from the push-draw syringe to the patient.

Various valve configurations and types of directional valve may be employed to perform the multi-stage delivery described above. In all versions of this invention, it is important that fluid first be delivered from a fluid source to a first syringe and then delivered sequentially to a second syringe. Ultimately, the fluid in the second, push-draw syringe is delivered sequentially to the patient. During each stage of the process, the source of fluid remains isolated from the patient. Typically, only one stage of the system operates at any given time.

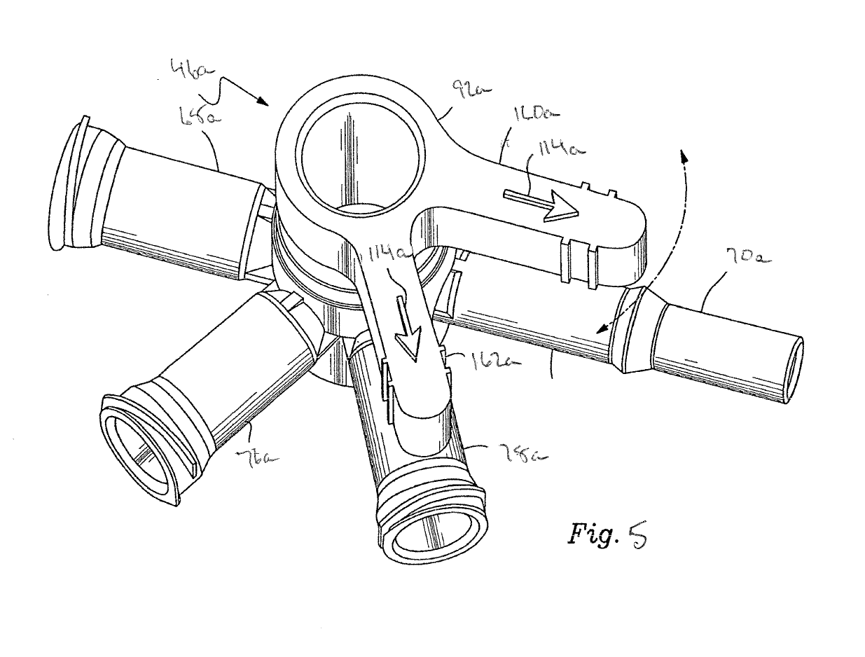

There is shown in FIG. 5 an alternative control valve assembly 46a, which again features a generally K-shaped valve body 66a composed of materials similar to those previously described. Aligned inlet and outlet conduit segments 68a and 70a, as well as transverse or angled conduit segments 76a and 78a are selectively interconnected to communicate and transmit fluid flow through respective pairs of the conduits by a rotatable stopcock valve analogous to that disclosed in the previous embodiment. In this version, the stopcock is rotated by a dual handle lever 92a, which includes elongate handles 160a and 162a. These handles 160a, 162a diverge from the hub of the stopcock lever at an angle of approximately 60 degrees, which matches the angle between each adjacent pair of fluid transmitting conduits 68a, 76a, 78a and 70a in the control valve assembly 46a. Each of the handles 160a and 162a is elongated and carries a respective directional arrow 114a that is printed, embossed or otherwise formed along the handle.

The valve lever 92a is turned to operate the stopcock (not shown) such that a selected pair of adjoining conduits is communicably interconnected to permit fluid flow therethrough. In particular, the stopcock is constructed such that the handles 160a and 162a are aligned with and extend along respective conduits that are communicably connected by the stopcock. In other words, the valve lever 92 is axially rotated until the handles 160a and 162a are aligned with adjoining conduits through which fluid flow is required. The angle between the handles 160a, 162a matches the angle between the adjoining conduits, e.g. 60 degrees. The lever 92a may therefore be rotated to align diverging handles 160a and 162a respectively with either conduits 68a and 76a, 76a and 78a, or 78a and 70a. In FIG. 5, the handles are aligned with conduits 78a and 70a, and arrows 114a point in a direction that is substantially aligned with those conduits. This indicates that the valve lever 92a is rotated and adjusted such that fluid is able to flow through the valve body 66a from the transverse conduit 78a to the outlet conduit 70a. The valve lever 92a is rotated to selectively align with the other pairs of conduits and thereby open the fluid flow passageway between the selected pair. The use of a dual handle valve lever 92a clarifies and facilitates usage of the control valve assembly 46a. Otherwise, the valve lever employed in the version of FIG. 5 is constructed and operates analogously to the valve lever disclosed in FIG. 1-3.

The use of multiple syringes is particularly critical and eliminates the risk of stacking that often occurs when a medical fluid is delivered under pressure directly from a source of fluid to a single delivery syringe. In that case, the syringe may be filled with fluid that exceeds the nominal volume of the syringe due to pressure stacking. If such fluid were to be delivered directly to the patient, this could result in a potentially dangerous overdose or fluid flooding. By transmitting the fluid from a reservoir syringe into a second, push-draw syringe, the pressure is equalized and only the fluid volume and pressure accommodated by the second syringe are delivered safely to the patient.

It is contemplated that the apparatus of the present invention be used in methods and procedures requiring delivery of medical gas. The following are examples of such applications:

CO₂ is useful in the following arterial procedures: abdominal aortography (aneurysm, stenosis) iliac arteriography (stenosis), runoff analysis of the lower extremities (stenosis, occlusion), renal arteriography (stenosis, arteriovenuous fistula [AVF], aneurysm, tumor), renal arterial transplantation (stenosis, bleeding, AVF), and visceral arteriography (anatomy, bleeding, AVF, tumor).

CO₂ is useful in the following venous procedures: venography of the upper extremities (stenosis, thrombosis), inferior vena cavography (prior to filter insertion), wedged hepatic venography (visualization of portal vein), direct portography (anatomy, varices), and splenoportograpy (visualization of portal vein).

CO₂ is likewise useful in the following interventional procedures: balloon angioplasty (arterial venous), stent placement (arterial, venous), embolization (renal, hepatic, pelvic, mesenteric) transjugular intrahepatic portacaval shunt creation, and transcatheter biopsy (hepatic, renal).

Angiography is performed by injecting microbubbles of CO₂ through a catheter placed in the hepatic artery following conventional hepatic angiography. Vascular findings on US angiography can be classified into four patterns depending on the tumor vascularity relative to the surrounding liver parenchyma: hypervascular, isovascular, hypovascular, and a vascular spot in a hypovascular background.

Improved CT colonography, an accurate screening tool for colorectal cancer, is performed using a small flexible rectal catheter with automated CO₂ delivery. This accomplishes improved distention with less post-procedural discomfort.

CO₂ gas is used as an alternative contrast to iodinated contrast material. The gas produces negative contrast because of its low atomic number and its low density compared with the surrounding tissues. When injected into a blood vessel, CO₂ bubbles displace blood, allowing vascular imaging. Because of the low density of the gas, a digital subtraction angiographic technique is necessary for optimal imaging. The gas bubble can be visible on a standard radiograph and fluoroscopic image.

CO₂ insufflation for colonoscopy improves productivity of the endoscopy unit.

Endoscopic thyroid resection involves creating a working space within the neck using CO₂ insufflation devices, with both axillary and neck approaches as starting points for dissection.

CO₂ insufflators are used during laparoscopic surgery.

Because of the lack of nephrotoxicity and allergic reactions, CO₂ is increasingly used as a contrast agent for diagnostic angiography and vascular interventions in both the arterial and venous circulation.

CO₂ is particularly useful in patients with renal insufficiency or a history of hypersensitivity to iodinated contrast medium.

CO₂ is compressible during injection and extends in the vessel as it exits the catheter.

CO₂ is lighter than blood plasma; therefore, it floats above the blood. When injected into a large vessel such as the aorta or inferior vena cava, CO₂ bubbles flow along the anterior part of the vessel with incomplete blood displacement along the posterior portion.

CO₂ causes no allergic reaction. Because CO₂ is a natural byproduct, it has no likelihood of causing a hypersensitivity reaction. Therefore, the gas is an ideal alternative. Unlimited amounts of CO₂ can be used for vascular imaging because the gas is effectively eliminated by means of respiration.

CO₂ is partially useful in patients with compromised cardiac and renal function who are undergoing complex vascular interventions.

Intranasal CO₂ is very promising as a safe and effective treatment to provide rapid relief for seasonal allergic rhinitis.

CO₂ is used for transient respiratory stimulation; encouragement of deep breathing and coughing to prevent or treat aterectasis; to provide a close-to-physiological atmosphere (mixed with oxygen) for the operation of artificial organs such as the membrane dialyzer (kidney) and the pump oxygenator; and for injection into body cavities during surgical procedures.

Medical asepsis is accomplished by using CO₂ in implant devices prior to surgical implantation. CO₂ may be effectively delivered to a foam generating tip for creating a medical foam for use in wound care and hair loss treatment.

In one embodiment, the present invention provides for an apparatus and use in a method whereby delivery of a gas alone is desired. The delivery of gas is independent of systems whereby a gas is delivered as a carrier for medications or other materials.

From the foregoing it may be seen that the apparatus of this invention provides for a system for safely delivering a controlled volume of a medical fluid to a patient and, more particularly to a system for delivery a controlled flow of CO₂ or other contrast media in order to obtain radiological images. While this detailed description has set forth particularly preferred embodiments of the apparatus of this invention, numerous modifications and variations of the structure of this invention, all within the scope of the invention, will readily occur to those skilled in the art. Accordingly, it is understood that this description is illustrative only of the principles of the invention and is not limitative thereof.

Although specific features of the invention are shown in some of the drawings and not others, this is for convenience only, as each feature may be combined with any and all of the other features in accordance with this invention.

While the invention has been described in its preferred form or embodiment with some degree of particularity, it is understood that this description has been given only by way of example, and that numerous changes in the details of construction, fabrication, and use, including the combination and arrangement of parts, may be made without departing from the spirit and scope of the invention.

1. A delivery system for the effective, reliable and foolproof delivery of controlled amounts of a medical grade gas to a patient, comprising
a compressed gas unit composed of a vacuum sealed bag to which is secured at least one compressed gas cartridge; and
a multi-part valve delivery system.

2. The delivery system according to claim according to claim 1, wherein the compressed gas cartridges are approximately 16 g to 45 g compressed gas cartridges.

3. The delivery system according to claim according to claim 1, wherein the compressed gas is medical grade CO₂ gas.

4. The delivery system according to claim according to claim 1, wherein the multi-part valve delivery system includes a flow control system.

5. The delivery system according to claim according to claim 4, wherein the flow control system includes an inlet conduit communicably joined to the compressed gas unit; an outlet conduit communicably joined to the patient; first and second syringes intermediate the inlet and outlet conduits; and a control valve assembly interconnecting the inlet conduit, the outlet conduit, the first syringe and the second syringe.

6. The delivery system according to claim according to claim 5, wherein the sealed bag includes a central cavity, an inlet port and an outlet port.

7. The delivery system according to claim according to claim 5, wherein the control valve assembly is alternatable between a first state wherein the inlet conduit communicates with the first syringe for transmitting fluid from the source to only the first syringe, a second state wherein the first syringe communicates only with the second syringe and is isolated from the inlet and outlet conduits for transmitting fluid from the first syringe to only the second syringe, and a third state wherein the second syringe communicates only with the outlet conduit and is isolated from the inlet conduit and the first syringe for transmitting fluid from the second syringe to only the outlet conduit.

8. The delivery system according to claim according to claim 7, wherein the control valve assembly includes a valve body having aligned inlet and outlet ports, the inlet port being communicably connectable to the inlet conduit and the outlet port being communicably connectable to the outlet conduit, the valve body further including a first intermediate port to which the first syringe is selectively connected and a second intermediate port to which the second syringe is selectively connected, the control valve assembly further including a stopcock element mounted rotatably within the body and including a channel consisting essentially of a first channel segment and a second channel segment, the first and second channel segments being selectively alignable with the inlet port and the first intermediate port to allow for communication between the inlet conduit and the first syringe, the first intermediate port and the second intermediate port to allow for communication between the first syringe and the second syringe, and the second intermediate port and the outlet port to allow for communication between the second syringe and the outlet conduit.

9. The delivery system according to claim according to claim 1, wherein the sealed bag includes a central cavity, an inlet port and an outlet port.

10. The delivery system according to claim 9, wherein the inlet port includes a cylinder cartridge puncture valve.

11. The delivery system according to claim 10, wherein the outlet port is connected to the multi-part valve delivery system.

12. The delivery system according to claim 1, wherein the sealed bag includes a loop allowing the sealed bag to be conveniently supported by a conventional medical stand.
